(19) United States
(12) Patent Application Publication
(10) Pub. No.: US 2021/0209816 A1
(43) Pub. Date: Jul. 8, 2021

(54) MODELLING PILEUP EFFECT FOR USE IN SPECTRAL IMAGING

(71) Applicant: MARS BIOIMAGING LIMITED, Addington (NZ)

(72) Inventors: Ali ATHARIFARD, Christchurch (NZ); Philip Howard BUTLER, Christchurch (NZ); Anthony Philip Howard BUTLER, Christchurch (NZ)

(21) Appl. No.: 17/059,537

(22) PCT Filed: May 30, 2019
(86) PCT No.: PCT/NZ2019/050060

§ 371 Date: Nov. 30, 2020

(30) Foreign Application Priority Data
May 30, 2018 (NZ) 742890

(51) Int. Cl. G06T 11/00; A61B 6/00; G01T 1/17; G06N 20/00; G06N 5/04

(52) U.S. Cl. CPC G06T 11/003; A61B 6/482; A61B 6/4241; G06T 2211/408; G06N 20/00; G06N 5/04; G01T 1/171

(57) ABSTRACT

Disclosed is a method for modelling a distorted energy spectrum in a multi-energy x-ray CT imaging system using probability distribution functions, the method including taking open beam measurements across a range of fluxes, estimating true interaction rates from said fluxes, calculating the probabilities of photons being counted, pileup order, and counts recorded to produce a model of the distorted energy spectrum. Also described are computer implemented methods and systems for modelling distortions and optimizing material images produced from multi-energy x-ray CT scanning systems using the modelling techniques.

### FIELD OF INVENTION

This invention relates to methods for addressing the distortion caused by pulse pileup in a multi-energy x-ray CT imaging system. More specifically, the invention relates to computer implemented methods for modelling the pileup effect in photon counting detectors in a multi-energy x-ray CT imaging system.

### BACKGROUND TO THE INVENTION

Multi-energy or spectral computed tomography (CT) is an x-ray imaging modality which produces 3D images of the inside of objects. In order to most effectively interpret the 3D images produced using this system, it is desirable for the images to be as clear as possible.

CT scanners use polychromatic x-ray sources which emit x-rays with various colours (x-ray energies). In regular CT there is no distinction made between the different energies of x-rays. However x-rays are absorbed differently by different materials in the body, and differently again for x-rays of different energies. Multi-energy CT measures the absorption of x-rays in different energy ranges. Using the differences in x-ray absorption in these energy ranges it is possible to classify and quantify various materials in an object, simultaneously.

An accurate classification of materials requires material-specific signatures, i.e. attenuation of the energy spectrum, to be measured accurately. Although photon counting detectors are capable of measuring much more spectral information than conventional energy integrating detectors, this information is rapidly distorted when photon counting detectors perform under high photon fluxes.

A major limitation of photon counting detectors is their limited count rate capability. Due to this limitation, such detectors may observe two or more almost simultaneous events as a single event with an energy different from the energy of the original photons. This phenomenon is called co-incident photon pileup effect or pileup effect, for brevity.

When the detector is exposed to high flux of photons, pileup effect modulates the energy spectrum by reducing the measured counts and distorting the recorded energy spectrum. Such spectral distortions need to be compensated for after the pileup effect is quantified through statistical calculations that account for photon emission, photon absorption and pulse detection processes.

Currently, known data reconstruction techniques do not account for pileup effects as the influence that this effect has on the measurements is not well-understood. To avoid degradations of spectral image quality due to the pileup effect, one option is to operate scanners at low photon fluxes. However, even at the low fluxes, there is still a chance of the photons being piled up, which means that the photon pileup effect is never removed and must be accounted for.

Another disadvantage of the current solution is increasing the scanning time which can be a limiting factor to the major applications of the spectral imaging. It would be advantageous to provide a method for accounting for pileup effects during data reconstruction to aid in the provision of accurate classification of materials in a multi-energy CT image.

A technique for calculating the pileup effect in a photon counting detector was introduced by Taguchi et al. (2010) that was then used by Taguchi et al. (2011), Cammin et al. (2014), and Wang et al. (2011) (see end of specification for full references). This technique has two major limitations: (i) estimating a realistic shape and width of the two (positive and negative) lobes of a pulse requires a good understanding of the pulse shape, and (ii) calculations are complex. Although pulse shape has been modelled for ideal simulation conditions (Ballabriga, 2009), the actual pulse shape is not clearly known as it can be adversely influenced by the electronics parameters and the experimental conditions. Since every pixel has separate readout electronics, analogue pulse shapes may vary across the pixel matrix. This uncertainty can affect accuracy of the pileup models. Applying these techniques to a commercial spectral scanner requires a significant amount of pre-measurement prediction before the scanning can take place, which often includes the need to run a large number of “pre-measurement” scans. This takes a significant amount of time and therefore results in added cost when spectral scanners are used for commercial purposes.

It would be an advantage to develop a method whereby the need for pre-measurement predictions is significantly reduced or removed from the process of obtaining images from a spectral scanner.

### OBJECT OF THE INVENTION

It is an object of the invention to provide a computer implemented method for modelling a distorted energy spectrum in a multi-energy x-ray CT imaging system using probability distribution functions.

Alternatively, it is an object of the invention to provide a method for optimising material images produced in a multi-energy x-ray CT imaging system.

Alternatively, it is an object to provide an improved method, system and/or computer programme for optimising the acquisition of spectral imaging data.

Alternatively, it is an object of the invention to at least provide the public with a useful choice.

### SUMMARY OF THE INVENTION

According to a first aspect of the invention, there is provided a method for modelling a distorted energy spectrum in a multi-energy x-ray CT imaging system using probability distribution functions, the method including the steps of:
a) taking open beam measurements across a range of fluxes;
b) determining detection model from flux measurements of step a);
c) estimating true interaction rate a from measurements made under various fluxes in step a);
d) measuring detector dead time τ;
e) calculating the probability of photons being counted using true interaction rate a and detector dead time τ from steps c) and d);
f) calculating the probability Prₘ of pileup order m using true interaction rate a and detector dead time t from steps c) and d);
g) calculating the probability of counts recorded across a range of energies (E) due to pileup order m; and
h) modelling the distorted energy spectrum based on the outputs from steps a) to g).

Preferably, the detection model of step (b) is a non-paralyzable or paralyzable detection model.

More preferably, the detection model is a paralyzable detection model.

Preferably, the step h) of modelling the distorted energy spectrum includes calculating the average number of counts observed across an energy range (Nₚᵤ) observed at a specific energy (E) by using the formula;

$${N}_{pu} ( E ) = a \times {t}_{exp} \times P {r}_{rec} ( a τ ) \times {\sum }_{m = 0}^{∞} [ {Pr}_{m} ( a τ , m ) \times Pr ( E , m ) ]$$

wherein (a) is the true interaction rate, tₑₓₚ is the exposure time, τ is the detector dead-time, Pr_{rec}(aτ) is the probability of events being recorded, Pr_{rec}(aτ,m) is the probability of pulse pileup order m, and Pr(E,m) is the probability of the counts recorded across the energy spectrum with mₜₕ order of pileup, wherein pileup of order m occurs when m+1 photons interact with a detector pixel within the same dead time period τ.

Preferably, step g) of calculating the probability of counts recorded across a range of energies Pr(E,m) due to pileup of different orders is achieved using the formula;

$$Pr ( E , m ) = ( \frac{1}{m + 1} ) [ {Pr}_{γ} ( E ) {★}^{( m )} {Pr}_{γ} ( E ) ]$$

wherein (m) represents the operation of convolution for m times.

Preferably, the method models one or more distortions in an energy spectrum due to a peak pileup effect.

According to a further aspect of the invention, there is provided a method for optimising images produced by a multi-energy x-ray CT imaging system, the method including the step of incorporating the method for modelling a distorted energy system as described above into a multi-energy x-ray CT imaging system and image reconstruction process.

Preferably the method of modelling a distorted energy system described above is incorporated into, or used in conjunction with a spectral image reconstruction algorithm and a material decomposition algorithm.

Preferably, the multi-energy x-ray CT system utilises three or more energy levels. More preferably, four to eight energy levels.

According to a further aspect of the invention, there is provided a computer implemented method for modelling a distorted energy spectrum in a multi-energy x-ray CT imaging system using probability distribution functions, the method including the steps of:
a) receiving open beam image measurements across a range of fluxes and storing said data on a data storage medium;
b) determining the detection model from measurements of step a);
c) estimating the true interaction rate a from measurements made in step a);
d) measuring a detector dead time τ;
e) calculating the probability of photons being counted using true interaction rate a and detector dead time τ from steps c) and d);
f) calculating the probability of pileup order m using true interaction rate a and detector dead time τ from steps c) and d);
g) calculating the probability of counts recorded across a range of energies (E) due to pile order m; and
h) modelling the probability of counts across the distorted energy spectrum based on the outputs from steps a) to g).

Preferably, the computer implemented method includes one or more further method steps as described above, when implemented on a computer system.

According to a further aspect of the invention there is provided a system for modelling a distorted energy spectrum in a multi-energy x-ray CT imaging system using probability distribution functions, the system comprising;
a) a means for receiving flat field image data generated at a range of tube current settings;
b) a means for storing the data;
c) a means for determining the detection model from measurements of step a);
d) a means for estimating the true interaction rate a from measurements made in step a);
e) a means for measuring detector dead time τ;
f) a means for calculating the probability of photons being counted using true interaction rate a and detector dead time τ from steps c) and d);
g) a means for calculating the probability of pileup order m using true interaction rate a and detector dead time τ from steps c) and d);
h) a means for calculating the probability of counts recorded across a range of energies (E) due to pile order m; and
i) a means for modelling the probability of counts across the distorted energy spectrum.

According to a further aspect of the invention, there is provided a computer implemented method for producing optimised material images in a multi-energy x-ray CT imaging system, the method including modelling a distorted energy spectrum in a multi-energy x-ray CT imaging system using probability distribution functions, the method for modelling including the steps of;
a) receiving open beam image measurements across a range of photon fluxes and storing data on a data storage medium;
b) determining the detection model from measurements of step a);
c) estimating the true interaction rate a from measurements made in step a);
d) measuring detector dead time τ;
e) calculating the probability of photons being counted using true interaction rate a and detector dead time τ from steps c) and d);
f) calculating the probability of pileup order m using true interaction rate a and detector dead time τ from steps c) and d);
g) calculating the probability of counts recorded across a range of energies (E) due to pile order m;
h) modelling the distorted energy spectrum based on outputs from steps a)-g);
wherein the method for producing optimised images further comprises;
i) operating a multi-energy x-ray scan under a high flux, to produce a set of scan data;
j) applying the model of step h) to the scan data produced at step i) to recover energy information and counts lost during acquisition of the scan data to produce a set of pre-processed data;
k) reconstructing spectral images from the pre-processed data in (j);
l) producing optimised material images.

Further aspects of the invention, which should be considered in all its novel aspects, will become apparent to those skilled in the art upon reading of the following description which provides at least one example of a practical application of the invention.

### BRIEF DESCRIPTION OF THE DRAWINGS

One or more embodiments of the invention will be described below by way of example only, and without intending to be limiting, with reference to the following drawings, in which:

FIG. 1 shows two charts (a) and (b), with (a) showing an illustration of Equation 6.3 as a function of the tube current for paralyzable and non-paralyzable detection models, and (b) the dead time loss approximated from 1−Pr_{rec}(aτ) for the two detector models;

FIG. 2 shows area plots of the probability of pileup orders for both paralyzable (2(a)) and non-paralyzable (2(b)) detection models;

FIG. 3 shows the true incident spectra (a) measured spectra (b) with a tube voltage of 80 kVp and five different tube current settings;

FIG. 4 shows the dead time loss in GaAs-Medipix3RX measurements, where due to the pileup effect, the dead time loss ratio increases when higher tube currents are used.

FIG. 5 shows variation of the recorded count rate with the true interaction rate. The detector dead time is approximated for the paralyzable and non-paralyzable detection models by fitting the statistical models (derived from Eq. 6.8) to the measurements;

FIG. 6 shows three spectra for tube voltage setting of 80 kVp in each plot: the scaled true incident spectrum, the energy spectrum modelled by pileup models according to the non-paralyzable detection model, and the measured spectrum using the Medipix3RX detector;

FIG. 7 shows three energy spectra for the tube voltage setting of 80 kVp: the scaled true incident spectrum, and the spectra with pileup orders m=0, 1, and 2 which are modelled by the pileup models according to the non-paralyzable detection model;

FIG. 8 shows three spectra for tube voltage setting of 80 kVp in each plot: the scaled true incident spectrum, the energy spectrum modelled by pileup models according to the paralyzable detection model, and the measured spectrum using the Medipix3RX;

FIG. 9 shows three energy spectra for the tube voltage setting of 80 kVp: the scaled incident spectrum, and the spectra with pileup orders m=0, 1, and 2 which are modelled by the pileup models according to the paralyzable detection model detector;

FIG. 10 shows a computer implemented method for modelling photon pileup effect in a multi-energy x-ray CT imaging system in one embodiment of the invention;

FIG. 10A shows a computer implemented method for optimizing scan parameters in a multi-energy x-ray CT imaging system in a further embodiment of the invention; and

FIG. 11 shows a representation of one embodiment of a system for implementing the method as shown in shown in FIG. 10.

### DETAILED DESCRIPTION OF PREFERRED EMBODIMENTS OF THE INVENTION

The use of multi-energy or spectral CT provides high spatial and energy resolution enabling the discrimination of different materials within an object. Spectral CT images enable identification of multiple different components within a single image. The clarity of the images produced is therefore very important, particularly in the medical field, where diagnosis and treatment methods are made based on the visual interpretation of what is seen in the produced images.

This invention describes a computer implemented method for using probability distribution functions to model a distorted energy spectrum caused by the pileup effect, in order to provide a pileup model that may be integrated into the data reconstruction process or work in conjunction with to optimise the scan/image output. As well as effecting a substantial decrease in scanning time, integration of these pileup models provides a dramatic improvement in the material contrast, which is a major objective of the spectral imaging.

For the purposes of this specification the method is performed using Medipix photon counting detectors (referred to as a detector going forward), but the method may be applied for use with other hybrid detectors known in the art. The method estimates the incident spectrum interacting with a sensor layer in a detector and measures the detector dead time, which indicates capability of the spectral CT camera in resolving the incident photons.

A series of probability functions for modelling the distortions caused by the pileup effect have been developed into pileup models customized for use with spectral CT cameras. When the scan parameters are known for a specific camera, the developed pileup models can model the count loss and distortion of the incident spectrum after the photons are processed through pixels electronics.

The developed pileup models can model the distortions due to the pileup prior to the scan. This can be beneficial in two ways: optimizing the scan parameters for acquiring high quality data, and integrating new information to the image processing chain to allow an advanced spectral image recovery.

Once the pileup models have been integrated into the processing chain, imaging can be undertaken under a higher flux, (an order of magnitude higher than what is used in current scans). For example, a count rate used in data acquisition by a scanning system of the present invention utilising a Medipix detector can increase from 4×10⁵ to 4×10⁶ count per second per square millimetre. The information derived from the pileup model is used to apply corrections/compensations to the data as a pre-processing step. The pre-processing step will recover misregistered energy information and counts lost in the acquisition of scan data due to the pileup effect. Recovered data is then fed into an image reconstruction process to produce a quantitative material image. With an accurate correction to the data made in the pre-processing step, there is no degradation in quality of either reconstructed or material images that are obtained from a material decomposition process.

The application of the pileup models in pre-processing of spectral data results generated under high flux results in the production of images of equal quality compared to images produced using a scan operating with a much lower flux, which take a significantly longer time (for example ten times longer) than scans performed under higher flux.

The term “high flux” or a “high flux rate” differs between different detectors being utilised, however, a “high flux” should be taken to mean any photon rate that can cause a statistically significant photon pileup in a detector. The exact photon rates may vary for various photon counting detectors, depending on their pulse processing capability. For example, in Medipix detectors, photon rates of about 4×10⁵ count per second per square millimetre are typical, with photon rates of 4×10⁵ count per second per square millimetre are considered as high flux.

“Pileup order” m occurs when m+1 photons interact with a detector pixel within one dead time interval, for example if two photons overlap within a dead time period, photon pileup of order 1 occurs and only one photon of a higher energy is registered.

The term “true interaction rate” is defined as a fraction of the photon flux that interacts with the sensor and contributes to the detector measurements.

Add any other definitions here used that aren't standard terms in the field.

Detection Models

The detector dead time or resolving time is defined as the time required between two consecutive photon events to be recorded as separate pulses. In general, following a photon interaction, the counting system is put in an inactive state for a finite period of time (the dead time). During this time, the deposited charge is collected and an analogue pulse with an amplitude corresponding to the energy of the photon event is created. All photons that are incident on the detector within the dead time period overlap and contribute to the shape of the observed pulse.

The counting mechanism of photon counting detectors is modelled either by paralyzable or non-paralyzable detection models. However, subtle effects often make such detectors behave somewhere in between.

In non-paralyzable detection models, after a photon turns the detector into the inactive state, the detector returns to the active state after a fixed dead time period τ. Therefore, the rate of incoming photons does not influence duration of the dead time in a non-paralyzable detection model.

In a paralyzable detection model, the detector dead time is not fixed, rather any additional photon arriving within the dead time of the previous event resets the dead time and keeps the detector in the inactive state for another t period.

Types of Pulse Pileup Effect

The pulses generated in the analogue circuit of the Medipix detectors are bipolar. In addition, the energy of the observed event is proportional to the height of the pulse generated in the analogue circuit. The overlap of the pulses can occur in two ways. Peak pileup occurs when two or more events interact with the detector within a dead time interval. In this case, a positive lobe of a pulse overlaps the main positive lobes of the previous pulses. Since the detector is in the inactive state, the two pulses cannot be resolved separately. Therefore, several events are observed as one, with a registered energy higher than the energies of the original photon events. The energy of the observed photon events may correspond to an energy above the x ray tube voltage (kVp). Therefore, peak pileup leads to the distortion of the recorded energy spectrum by losing counts and adding a high energy tail.

Another type of pileup effect occurs when a positive lobe of a pulse overlaps the negative tails of the previous pulses. This effect is called tail pileup in which the overlaps can cause the energy of the photons to be observed slightly lower than their original energies. Therefore, tail pileup may add a small low energy tail to the measured spectrum. It should be noted that although tail pileup distorts the energy spectrum, it may not influence the number of counts.

Factors Influencing Pileup Effect

The pulse pileup effect can be more or less significant depending on the pulse processing capability of the detector, exposure settings, and the geometry and setup of the imaging system. A shorter dead time enables the pulse processing circuit to quickly resolve and register the incident photon events and reduce the chance of the events overlaps. In the Medipix detectors, dead time can vary with the shaping time and within the process of compensating the leakage current. It can also vary according to the energy of the incident x-ray photon and the threshold level used for imaging.

True interaction rate: in order to study and understand the influence of the photon pileup effect, the spectrum that interacts with the sensor layer needs to be known. The true interaction rate is defined as the fraction of the photon flux that interacts with the sensor and contributes to the detector measurements. A higher interaction rate requires a faster photon processing circuit that allows resolving a photon before a subsequent photon interacts with the sensor. The true interaction rate is energy dependent and varies based on the quantum detection efficiency of the sensor and the filtration used for the measurements. These two factors can modulate the polychromatic source spectrum and reduce the number of photons that interact with the sensor.

A filter attenuates the source spectrum and reduces the number of photons that reach the detector, therefore reducing the chance of photons to be piled up. For instance, a 4 mm aluminium filter reduces the true interaction rate more than a 1 mm aluminium filter.

Pulse shape: the analogue pulses have finite widths. In addition to the positive lobe of a pulse that corresponds to the energy of an interacting photon, a pulse typically has a relatively long negative tail (undershoot) that returns to the baseline long after the dead time ends. In the Medipix chip, the time for pulse shaping is controlled by the Ikrum bias current that is sent to the pixels globally. This current is adjusted via 8-bits and adversely affects the shaping time and the pulse height. The preamplifier return to zero is also controlled by the Ikrum current. Therefore, the Ikrum setting can affect the count rate capabilities and the gain of the chip. Increasing Ikrum can contribute to the system noise. Choosing an optimal setting for Ikrum is essential and is operated by the chip designers. In case of changing the bias voltage, Ikrum setting needs to be re-optimized to cancel the leakage current.

In real implementation of the Medipix chip, the pulse undershoot originates from a slow decay determined by the pre-amplifier's feedback network. The decay of the pre-amplifier output is differentiated in the shaper, which generates an undershoot in the shaper's output. A pole-zero cancellation technique used in the design of the Medipix chip effectively eliminates the pulse undershoot. This technique adds a zero in the shaper transfer function to cancel the pole associated with the decay in the output of the pre-amplifier. Therefore, the tail pileup effect is expected to be negligible in the Medipix detectors.

Pileup Models

This section introduces analytical models for simulating the distortions caused by the pileup effect in Medipix detector measurements which are the focus of this invention. The previous section discussed that the pulse tails are effectively eliminated using the pole-zero cancellation technique.

Therefore, the models developed in this section only consider distortions due to the peak pileup effect. However, the following sections will discuss the experimental results to investigate possible distortions in the measurements due to the tail pileup effect.

Different models are introduced for paralyzable and nonparalyzable detection models.

To model the distortions caused by the pileup effect, several probability density functions are taken into account. The calculations of the distorted recorded spectrum are formulated as Equation 6.2 below:

$${N}_{pu} ( E ) = a \cdot {t}_{exp} \cdot {Pr}_{rec} ( a τ ) \cdot {\sum }_{m = 0}^{∞} [ {Pr}_{m} ( a τ , m ) \cdot Pr ( E , m ) ]$$

where Nₚᵤ(E) is the average number of counts observed across the energy range. a is the true interaction rate, tₑₓₚ is the exposure time, Pr_{rec}(at) is the probability of events being counted, Prₘ(aτ,m) is the probability of pulse pileup order m, and Pr(E,m) is the probability of the counts recorded with energy E due to the pileup of m^{th} order. The true interaction rate (a) and the detector dead time (τ) are measurable when the MARS camera is operated under certain experimental conditions. The exposure time is selected by the user at the data acquisition time. The detector dead time and the true interaction rate are approximated from the experimental measurements (see experimental examples below). Analytical derivations of the probability functions are presented below.

Probability of Events being Counted

The recorded count rate can be analytically obtained from the probability of time intervals between the adjacent photon events. The ratio of the recorded count rate, aᵣ, to the true interaction rate, a, is a function of the true interaction rate and the dead time of the detector. The probability of events being counted is defined as Equation 6.3;

$${Pr}_{rec} ( a τ ) = \frac{{a}_{r}}{a}$$

where the product of aτ gives the average number of photons that interact with the detector within a dead time period. The calculations of this probability for the two detection models are discussed in Knoll (2010). Below, a brief description of the calculations for the two detection mechanisms is presented. For non-paralyzable detection models, the duration of the interval within which the detector is the inactive state is given by aᵣr. Within this time period, events are lost at the rate of aaᵣτ. On the other hand, the rate of loss could be expressed as a-aᵣ, therefore

a−aᵣ=aaᵣτ (Eq. 6.4)

Solving for aᵣ, and dividing the two sides of the equation by a gives Equation 6.5:

$${Pr}_{rec} ( a τ ) = \frac{1}{a τ + 1}$$

This is not applicable to the paralazable detectors where the duration of the inactive state is not fixed. The distribution function of the intervals between random photon events that interact at an average rate of a is (Eq. 6.6)

P(t)dt=ae^{-at}dt

where P(t)dt is the probability of observing an interval whose length lies within dt. For a paralyzable detector, the rate of observing the incoming events is equal to the probability of time intervals between two successive events when they arrive at a time difference larger than τ.

Therefore, the probability of the intervals that are larger than the dead time are obtained by integrating Eq. 6.6 between r and infinity as shown below (Eq. 6.7)

$$\begin{matrix}{Pr}_{rec} ( aτ ) = {\int }_{τ}^{∞} {ae}^{- at} dt \\ = {e}^{- aτ}\end{matrix}$$

Eq. 6.8 summarizes the probability of counting the incoming events for the two detection models:

$$P {r}_{r e c} ( a τ ) = \{ \begin{matrix}\frac{1}{a τ + 1} & Non paralyzable model \\ {e}^{- a τ} & Paralyzable model\end{matrix}$$

Dead time loss: The dead time loss is expressed as the rate of losing counts due to overlap of events within the detector dead time. It can be expressed as the percentage of the events that are not counted. Therefore, the dead time loss (DL) can be obtained from Eq. 6.8 as Eq. 6.9;

DL=[1−Pr_{rec}(aτ)]×100[%]

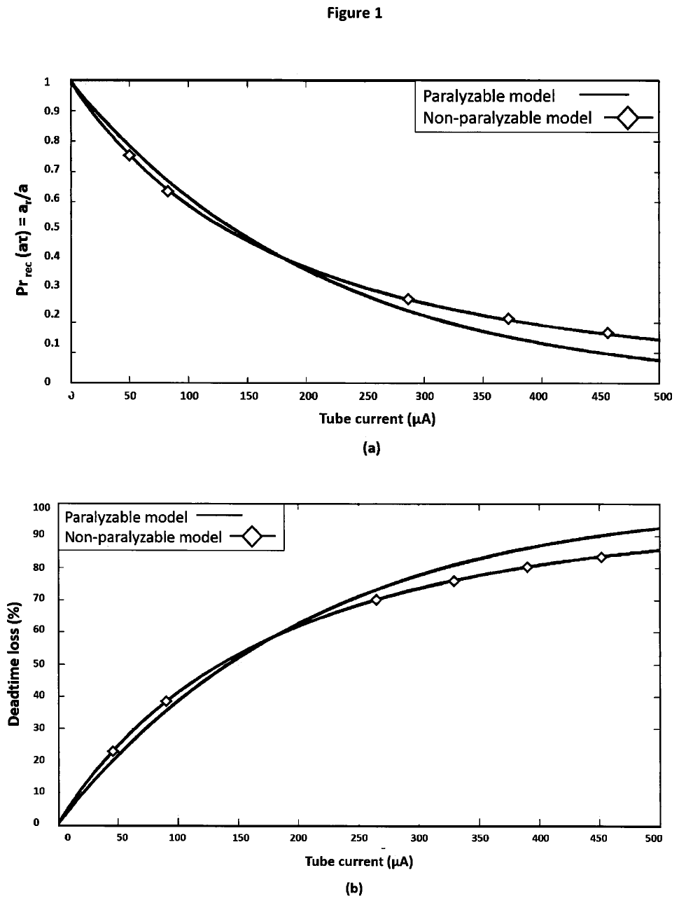

Referring to FIG. 1, this shows two charts (a) and (b), with (a) showing an illustration of Eq 6.3 as a function of the tube current for paralyzable and non-paralyzable detection models, and (b) the dead time loss approximated from 1−Pr_{rec}(aτ) for the two detector models.

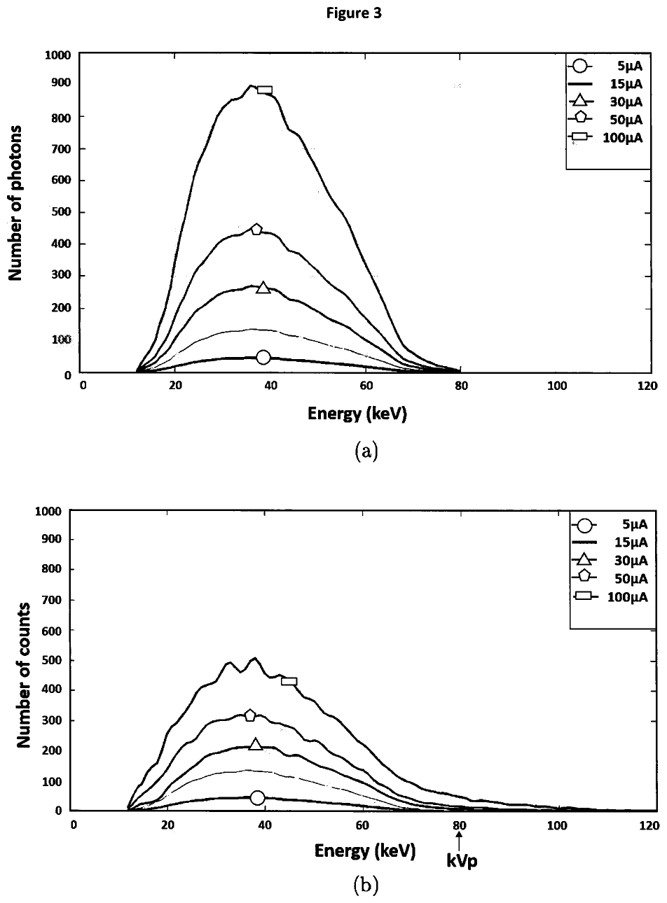

It should be noted that the diamond indication marks on the non-paralyzable model in FIG. 1 are merely differentiating the line form the paralyzable model and are not an indication of specific data values. This also applies to FIGS. 3, 5, 7 and 9 in that the shape indicators are to differentiate lines on the chart, rather than representing specific data points.

FIG. 1 (a) illustrates the probability of events being counted, as calculated in Eq. 6.8. Note that the probability for the two detector models are similar at low true interaction rates where the average number of events interacting with detector within the dead time is very small (aτ<<1). By increasing the tube current, and correspondingly the photon flux, the average number of events (aτ) that interact with the detector also increases. As a result of this, there will be a higher chance of photon overlap, thus lower probability of photons being recorded.

FIG. 1 (b) illustrates the dead time loss for the two detection models. The dead time loss ratio for the non-paralyzable detector model is slightly higher at lower tube currents. This is because of higher product of aτ for the non-paralyzable model that is justified by higher a estimated for this model than for the paralyzable model. However, having a higher count loss at higher fluxes for a paralyzable model is in agreement with the definition of this model that loses counts due to its updating dead time.

Probability of Pileup Orders

Prₘ(aτ,m) in Eq. 6.2 is the probability of overlapping m events that interact with the detector within a dead time interval. Since the detector dead time is defined differently for the two detection models, a separate calculation of this probability is discussed for each model. For a non-paralyzable detector, the probability is calculated from the poisson distribution. The average number of the photons that interact with the detector is aτ. According to the poisson distribution, the probability of having m events within the dead time τ is given as (Eq. 6.10)

$$P {r}_{m} ( aτ , m ) = \frac{{( a τ )}^{m} {e}^{- a τ}}{m !}$$

where m is the pileup order. Note that a pileup of mth order occurs when m+1 photons interact with the detector within the same dead time period. Therefore, m=0 indicates no overlap of events.

To derive Prₘ(aτ,m) for the paralyzable detection model, let m=1, assuming that two and only two photons overlap. Given the first event arriving at t=0, the second event arrives in the interval 0<t<τ, and the third event arrives after a t interval from the second event. The former can be calculated from the probability of time intervals (Eq. 6.6) between zero and t, and the latter is the same as Eq. 6.7.

Therefore (Eq. 6.11);

Prₘ(aτ,1)=∫₀^{τ}ae^{-at}dt×e−^{aτ}=(1−e^{-aτ})×e^{-aτ}

Extending this probability to higher orders of pileup, the first component in Eq.6.11 is repeated for m times as each of the first m events interacts with detector within t of the previous event. However, the second component remains the same as the time interval between (m+1)th and (m+2)th events is larger than t. Therefore, for a paralyzable detector, the probability of the observed counts being made of m+1 photon events is given as;

$$\begin{matrix}P {r}_{m} ( aτ , m ) = {( 1 - {e}^{- a τ} )}^{m} \times {e}^{- a τ} & ( 6.12 ) \\ where & \\ \begin{matrix}{\sum }_{m = 0}^{∞} P {r}_{m} ( aτ , m ) = {e}^{- a τ} {\sum }_{m = 0}^{∞} {( 1 - {e}^{- a τ} )}^{m} \\ = {e}^{- a τ} {e}^{a τ} = 1\end{matrix} & ( 6.13 )\end{matrix}$$

The following equation assesses the validity of Eq. 6.12. Since Prₘ(aτ,m) corresponds to overlap of m+1 true events, the average number of true events per count can be calculated as the following:

$$\begin{matrix}{\sum }_{m = 0}^{∞} ( m + 1 ) {Pr}_{m} ( aτ , m ) = {e}^{- aτ} {\sum }_{m = 0}^{∞} ( m + 1 ) {( 1 - {e}^{- aτ} )}^{m} \\ = {e}^{- aτ} {e}^{2 aτ} \\ = {e}^{aτ}\end{matrix} \qquad ( 6.14 )$$

The reverse of this expression is consistent with the result obtained from Eq. 6.7, which means every photon event is accounted for. Eq. 6.15 summarizes the probability of mth order pileup event for the two detection models,

$$P {r}_{m} ( aτ , m ) = \{ \begin{matrix}\frac{{( a τ )}^{m} {e}^{- a τ}}{m !} & Non P aralyzable model \\ {( 1 - {e}^{- a τ} )}^{m} \times {e}^{- a τ} & Paralyzable model\end{matrix} \qquad ( 6.15 )$$

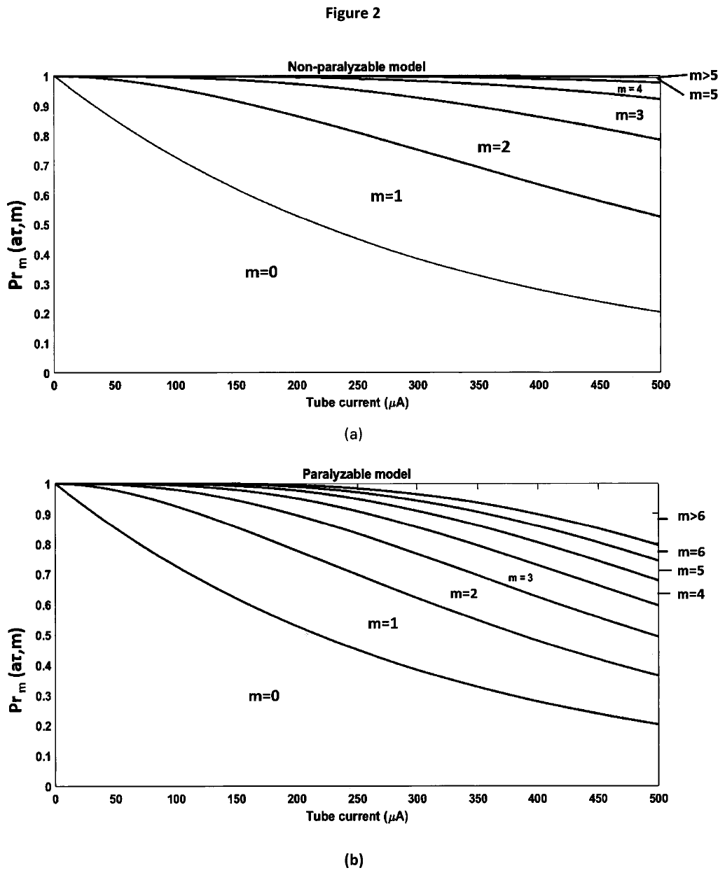

FIG. 2 illustrates area plots of the probability of pileup orders for both paralyzable and non-paralyzable detection models. For both models, probability of events piled up with the higher orders increases with the tube current. FIG. 2 (a) shows that up to IA=500 μA, pileup effect of orders m 6 has almost no contribution to the modelled distorted spectrum. FIG. 2 (b) illustrates that these orders have a significant contribution to the modelled spectrum at higher tube currents for the paralyzable model. This is consistent with the expectations from the paralyzable detection model due to overlap of more photon events at higher fluxes.

Probability of the Counts Recorded Across the Energy Spectrum

The MARS spectral scanner uses a polychromatic x-ray tube that generates photons with energies of up to 120 keV. When the MARS camera is exposed to the x-ray beams, photons of various energies can overlap. The energy of the observed photon may vary depending on the energy of the original photons and the time interval between them. Given the same peaking time for the photons of different energies, if two overlapping photons arrive exactly at the same time, the detection system will observe a photon with an energy equivalent to the sum of original events energies. The energy of the observed photon decreases with increasing the time interval between the photon events.

Since the counting mechanisms of paralyzable and non-paralyzable detection models are different, the rate of the decrease in the observed energy is expected to be different. However, the difference of the observed energy can be neglected particularly when the effect of low orders of pileup is dominant in the measurements. Therefore, identical calculations for the probability of the counts that are recorded at every energy E across the spectrum with the two detection models are used here.

A realistic calculation of the observed energy is complex and requires a sufficient understanding of several factors such as the pulse shape and the peaking time respective to photons of different energies. The current study assumes that while the detector is in the inactive state, the energy of the observed event is equivalent to the sum of the energies of all co-incident events.

The probability distribution function of the photon energy across the energy spectrum is given by

$$P {r}_{γ} ( E ) = \frac{{S}_{0} ( E )}{{\int }_{0}^{∞} {S}_{0} ( E )} \qquad ( 6.16 )$$

where S₀(E) is the true incident spectrum, that is the x-ray spectrum that interacts with the detector. Note that this probability is normalized, R∞0Pr_{y}(E)=1. The distribution of the sum of two independent random variables can be calculated as the convolution of the two variables distributions. Using this principle for randomly distributed polychromatic x-ray spectra, the probability distribution function of counts at the energy E with the mth order of pileup, Pr(E,m), is obtained,

$$P r ( E , m ) = ( \frac{1}{m + 1} ) [ P {r}_{γ} ( E ) {★}^{( m )} {Pr}_{γ} ( E ) ] \qquad ( 6.17 )$$

Where *^{(m)} represents the operation of convolution for m times. A factor of [1/(m+1)] is used to account for the overlap of the events due to the peak pileup effect. The substitution of Eq. 6.8, Eq. 6.15, and Eq. 6.17 into Eq. 6.2 can provide the simulation of the measured spectrum distorted by pulse pileup effect in the MARS scanner.

In variations of the model, the last component of the model (Pr(E,m))—i.e. probability of the counts recorded with energy E due to the pileup of m^{th} order, may be calculated in a more realistic form. This component assumes that the analogue signals generated in the front-end electronics are delta pulse functions. However, every pulse is a bipolar pulse with a certain width. The energy of the overlapped pulses can be more accurately modelled if the pulse shape corresponding to photons of various energies are known. Currently, this cannot be measured on a per event basis. However, when measured, this information can be incorporated into the models to better model the energy of the photon that is observed as a result of overlapping a number of photon events.

Experimental Evaluation

An evaluation and quantitative analysis of the pileup models is outlined below, with measurements made by a GaAs-Medipix3RX detector in a spectral CT scanner.

A Medipix3RX chip flip-bonded to 0.5mmGaAs sensor is used. Prior to the data acquisition, threshold equalization and energy calibration were performed. A −300V bias voltage was applied to the sensor electrodes to allow fast collection of charge within the thin layer of GaAs sensor. Several datasets were acquired by sweeping the threshold values. The experimental settings are listed in Table 6.1 below. 100 measurements were made at every 1 keV energy threshold starting from 12 keV up to twice as the x-ray tube voltage. The measurements were averaged and the differential recorded spectrum was obtained by subtraction of the average counts between the adjacent energy thresholds. The differential spectrum was then smoothed.

Several tube currents were used to allow observation of various distortion levels of the pileup effect. Note that for the current operational setup, the maximum number of counts that each counter of Medipix3RX pixels can record is limited to 4095. In order to avoid saturation of the detector, the exposure time was decreased proportionally to every increase in the tube current.

TABLE 6.1
Experimental parameters for energy response
measurements at different fluxes

| Detector | Detector GaAs-Medipix3RX |
|---|---|
| Operation mode | Spectroscopic-CSM |
| Pixel pitch (μm) | 110 |
| Filtration (mm) | 1.8 Al inherent (equiv.) |
| Source to detector distance (mm) | 186.9 |
| Energy threshold (keV) | increments within 12-160 |
| Tube voltage (kVp) | 80 |
| Exposure time (ms) | 400, 133, 67, 40, 20, 10, 5 |
| Tube current (μA) | 5, 15, 30, 50, 100, 200, 400 |
| Number of measurements | 100 |

The measured spectra were then scaled up to allow a comparison between several sets of measurements.

Approximation of the True Count Rate

The true interaction rate can be obtained from source spectrum models such as SpekCalc (Poludniowski et al., 2009) or MARS source model (Shamshad, 2017), Monte Carlo calculations (Taguchi et al., 2010), or experimental measurements (Taguchi et al., 2011; Koenig et al., 2013).

During this study, it was observed that the modelled spectrum (after the corrections are applied for filtration and sensor's quantum detection efficiency) may not accurately resemble the true spectrum that is expected in an actual experimental condition. In this section, the true interaction rate is obtained from the first set of measurements that use IA=5 μA. This is the lowest possible value that the spectral scanning user interface allows to be set for the x-ray tube current. Since the photon flux is linearly proportional to the tube current, the true interaction rates that correspond to higher tube currents are estimated by proportional scaling of this rate.

Dead Time Measurement

Different techniques for estimating detector dead time are known. Commonly known techniques consider non-linearity of the measured count rate to the photon flux.

A typical example is a two-source method that uses two x-ray sources individually and in combination. The observed count rate from the combined sources would result in the observation of a lower count rate than the sum of the count rates observed when the sources are used individually. Then, the dead time can be calculated from the discrepancy between the observed rates. Since no approximation was considered for calculating the probability of events being counted, the detector dead time in this study is approximated by fitting the measurements to this probability. Note that in this probability (as defined in Eq. 6.8), the recorded count rate and the true interaction rate are known, and the only unknown variable is the detector dead time.

Methods for Evaluation and Quantitative Analysis

In order to evaluate the developed pileup models, three different spectra were compared. The true incident spectrum (also called scaled incident spectrum), the energy spectrum modelled by the pileup models for either paralyzable or non-paralyzable detection mechanism, and the spectrum measured by the GaAs-Medipix3RX detector.

The motivation is to investigate capability of the models in simulating the experimental measurements. For the quantitative analysis of the agreement between the results of the models and the measurements, the root mean square difference (RMSD) and the coefficients of variation (COV), also called relative standard deviation, were calculated over the energy range of 20 keV-130 keV.

$$\begin{matrix}R M S D = \sqrt{{\sum }_{E} [ {N}_{R} ( E ) - {N}_{x} ( E ) ] / n} & ( 6.18 ) \\ and & \\ COV = R M S D / ( {\sum }_{E} {N}_{R} ( E ) / n ) \times 100 [ \% ] & ( 6.19 )\end{matrix}$$

where N_{R}(E) and Nₓ(E) are vectors of the same size. N_{R}(E) is the vector of the recorded counts, containing the measurements at every 1 keV energy threshold. Nₓ(E) can be either the spectrum modelled by the models (Npu(E)) or the scaled incident spectrum (scaledS₀(E)), and n is the number of sampling points. In the case of the analysis provided in this section, n=130−20+1=111.

Experimental Analysis

The true interaction rate at I_{A}=5 μA was measured to be 7.9×103 cps/mm2. FIGS. 3 (a) and (b) illustrates the estimated true incident spectra and the measured spectra with different tube current settings. This figure shows that the measured spectrum with the tube current of 5 μA ends at the selected tube voltage (80 kVp) that confirms negligibility of the pileup effect at this tube current. However, a high energy tail is added to the measured spectra when higher tube currents are used. Apart from the high energy tail, the measured spectra in FIG. 3(b) show lower height than true incident spectra in FIG. 3(a). This is due to the count loss in the observed spectra as a result of overlapping the co-incident photons. As the tube current increases, the pileup effect causes a more significant discrepancy between the measured spectra and the true incident spectra.

If the negative pulse tails are not eliminated, tail pileup effect can lead to the observation of a photon event at a lower energy than its original energy. A similar rather more severe effect could appear due to the charge sharing effect. There is no low energy tail observed in the GaAs Medipix3RX measurements (see FIG. 3b). This illustrates that the pulse tails have been efficiently suppressed by the pole-zero cancellation circuit implemented in the Medipix3RX chip. In addition, this confirms the effectiveness of the charge summing and allocation architecture of the chip that mitigates the charge sharing effect.

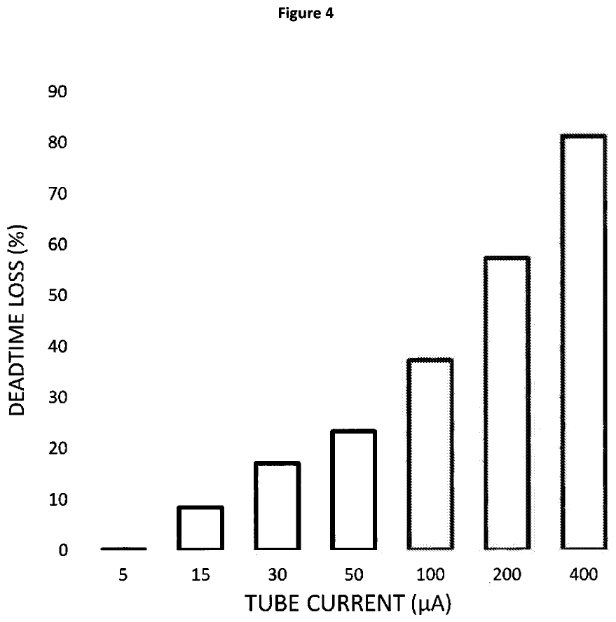

When operated under very high fluxes, other than pileup effect, the performance of photon counting detectors deviates from theory due to additional factors such as ASIC power, and charge sharing effect. Under such circumstances, the detector count rate can no longer be linearised by compensation techniques. Due to this, the nuclear medicine community does not usually accept the quality of the data when the dead time loss exceeds 30-40%. FIG. 4 illustrates that in the measurements, the dead time loss increases as the true interaction rate increases and it reaches 81.17% at the tube current of 400 μA. The analysis presented in this section uses the measurements made with tube currents of up to IA=100 μA at which the dead time loss is 37.2%.

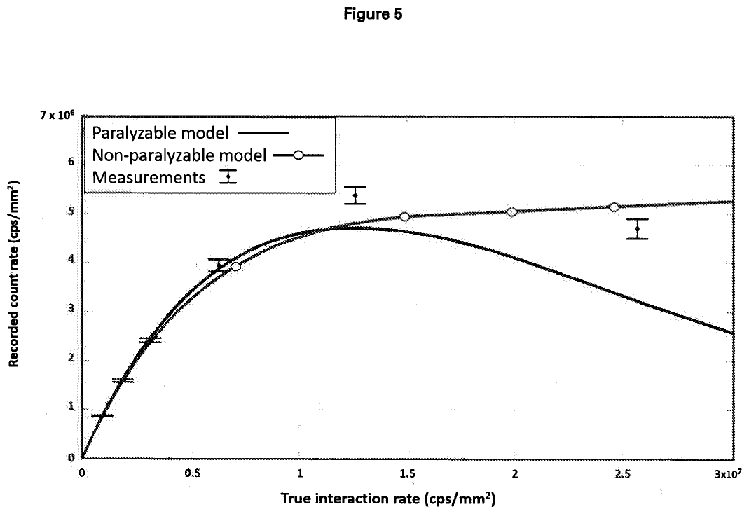

FIG. 5 illustrates the recorded count rate as a function of the true interaction rate. Also, it shows the count rate behaviours of the two detection models. While a non-paralyzable detector cannot record counts above a certain rate, the count rate of a paralyzable detector begins to drop with increasing the true interaction rate as more number of counts are lost due to the updating dead time. This figure shows that the detector count rate undergoes a falling trend at high true interaction rates.

As illustrated in FIG. 5, the dead time of the GaAs-Medipix3RX detector was estimated by fitting the probability of events being counted to the measurements. The mean and standard deviation of the detector dead time was estimated to be τ=4.21±0.33 μs and τ=5.56±0.41 μs for the paralyzable and non-paralyzable detection models, respectively. Note that the following analysis assumes the same true interaction rate for the two detection models.

Evaluation and Quantitative Analysis

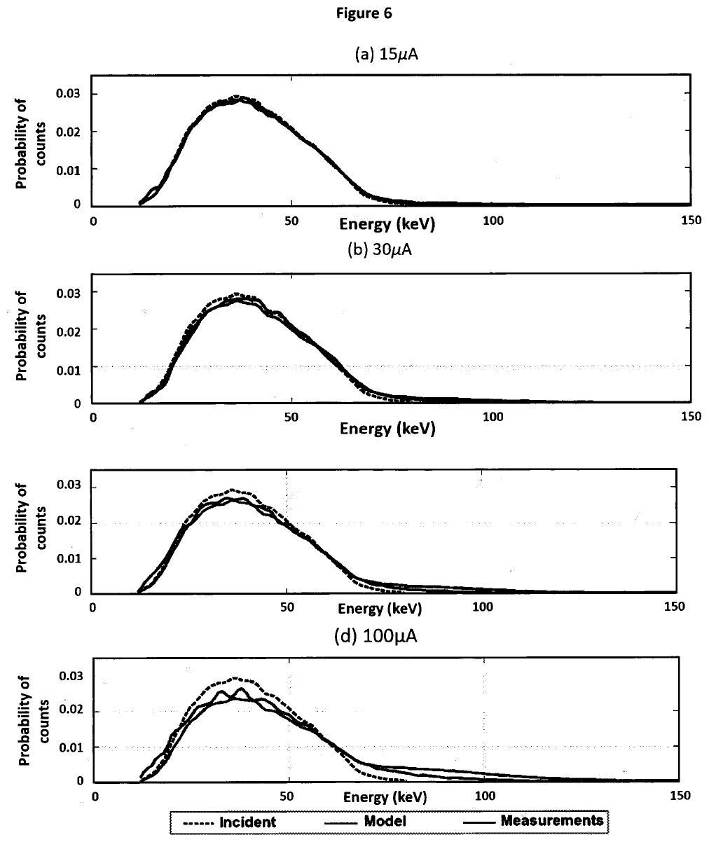

FIG. 6 illustrates the evaluation results when the measurements are compared with the simulations of pileup models according to the non-paralyzable detection models. Each plot compares the results for a different tube current. Each plot includes three curves: (1) the true incident spectrum, (2) the spectrum measured using the GaAs Medipix3RX detector, and (3) the spectrum modelled by the pileup models for the non-paralyzable detectors. The probability distributions of the counts are plotted to allow a visual comparison of the results for different tube currents. The simulations of the models agreed reasonably well with the measurements. The pileup tail is modelled by the models very well when low tube currents are used (see FIGS. 6a, and 6b.

However, a discrepancy begins to appear between the modelled spectrum and the measured spectrum when the tube current increases. In particular, for the tube current of IA=100 μA in FIG. 6d, pileup tail is overestimated by the models whereas the bremsstrahlung spectrum is underestimated.

FIG. 6 illustrates the contribution of the pileup orders to the modelled spectra shown in FIG. 6. For all four tube current settings, the zero-th order of pileup remains within the incident spectrum. Also, this figure shows that the discrepancy between the measured spectra and the modelled spectra in FIG. 6 is due to an underestimation of the zero-th order and an overestimation of the higher orders of pileup, in this case mainly the first order.

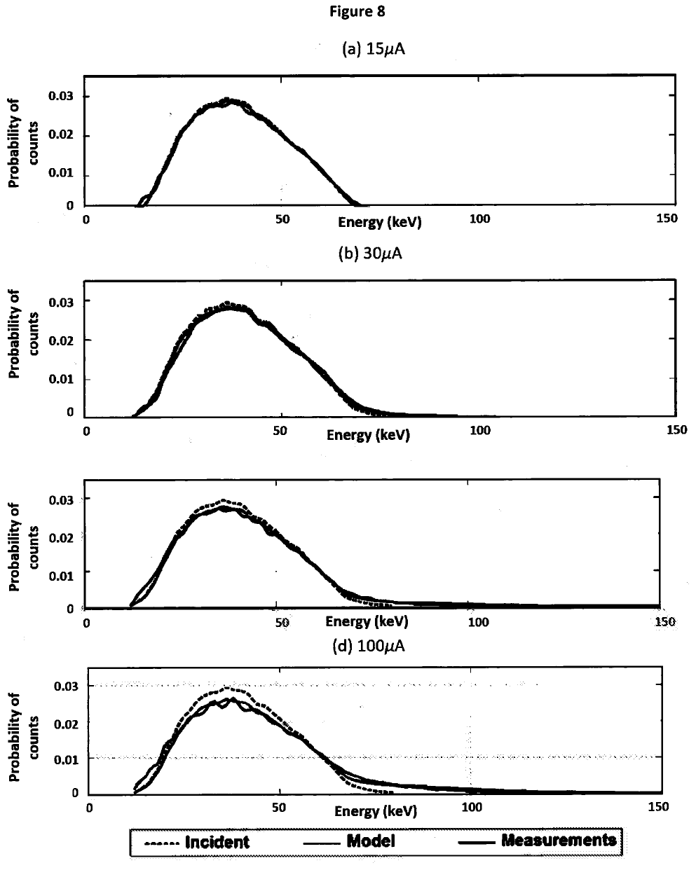

FIG. 8 illustrates the evaluation results for the case that the measured spectra are modelled by the pileup models according to the paralyzable detection models. The modelled spectra are compared with the measured spectra and the estimated true incident spectra for four different tube currents. It is apparent that the paralyzable models could simulate the recorded energy spectra much better than the non-paralyzable models. This is particularly evident at energies above the tube voltage where the pileup tail appears in the measured spectrum (compare FIG. 8 with FIG. 6).

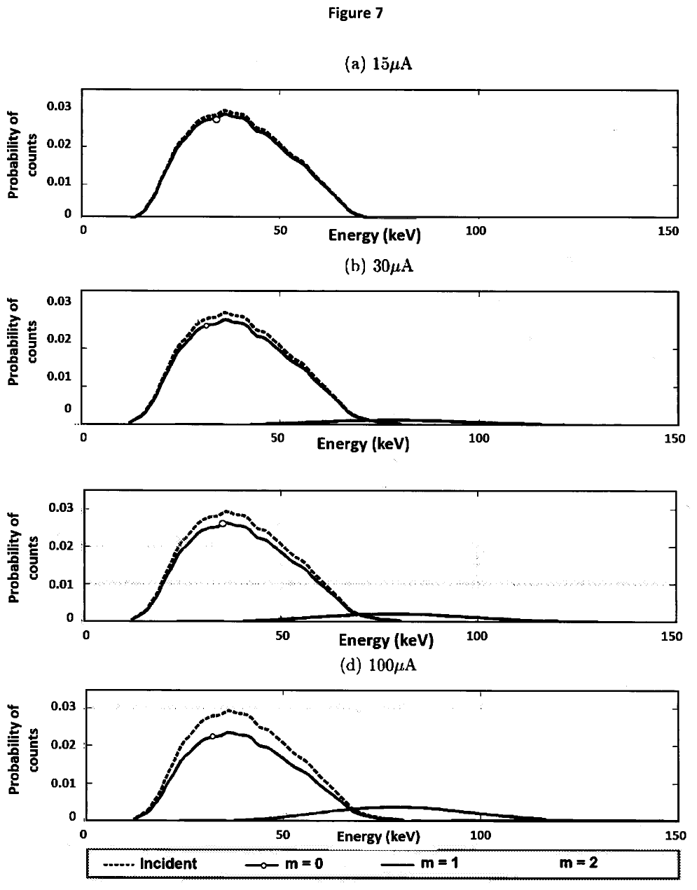

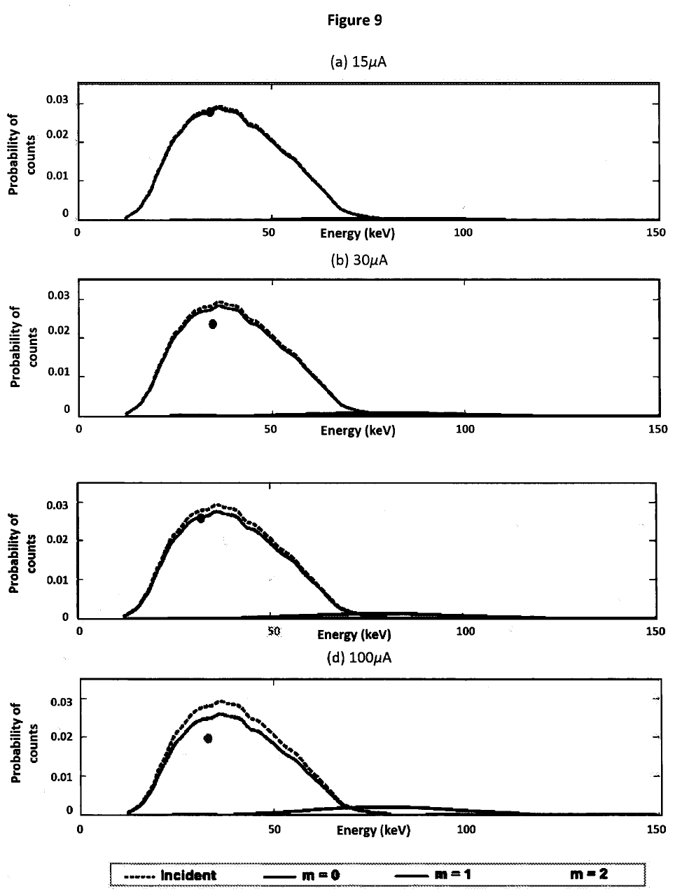

FIG. 9 shows that a stronger zero-th order and a smaller contribution of higher orders (especially the first order) have enabled the paralyzable model to model the measurements better than the non-paralyzable model (compare FIG. 9 with FIG. 7). This is consistent with the probability of pileup orders that was previously illustrated in FIG. 2.

Table 6.2 below summarizes the quantitative analysis of the modelled spectra for all pixels.

TABLE 6.2
RMSD and COV calculated against the spectra measured
by a GaAs-Medipix3RX detector. Dead time loss and
the average number of photon interactions within
a dead time interval (ατ) are also included.

| | | Tube Current [ατ] (deadtime loss) | | | |
|---|---|---|---|---|---|
| | | 15 μA | 30 μA | 50 μA | 100 μA |
| | | [0.05] | [0.1] | [0.16] | [0.32] |
| | Scheme | (8.47%) | (17.1%) | (23.3%) | (37.2%) |
| RMSD | N_{R}(E), scaled S₀(E) | 1.8 | 14.0 | 39.5 | 149.6 |
| [counts] | N_{R}(E), N P_{model}(E) | 1.69 | 5.58 | 13.38 | 43.30 |
| | N_{R}(E), P_{model}(E) | 3.25 | 5.58 | 6.00 | 15.16 |
| COV | N_{R}(E), scaled S₀(E) | 4.9 | 20.9 | 38.9 | 90.4 |
| [%] | N_{R}(E), N P_{model}(E) | 4.6 | 8.3 | 13.2 | 26.2 |
| | N_{R}(E), P_{model}(E) | 8.9 | 8.3 | 5.9 | 9.2 |

The RMSD and COV were calculated between the measured spectrum (N_{R}(E)), and the scaled true spectrum (scaledS₀(E)) that is the true incident spectrum. These measures were also calculated between the measured spectrum (N_{R}(E)) and the spectrum modelled by the pileup models that were developed according to the paralyzable and non-paralyzable detection models (P_{model}(E) and NP_{model}(E), respectively). In general, at all different tube current settings, the distorted spectra modelled by both detection models were much closer to the measured spectrum than the true incident spectrum.

According to Table 6.2, RMSD and COV are equal when the two models are compared with the measurements at IA=30 μA. However, these measures are much higher for the non-paralyzable models at higher tube currents. For example, the RMSD and COV between the paralyzable models and the measurements remain below 15.16 and 9.2% respectively for the tube currents of up to 100 μA where 37.2% of the counts are lost. The only exception is at IA=15 μA where RMSD and COV between the paralyzable models and the measurements are slightly higher than those between the non-paralyzable models and the measurements. This can be due to the approximation of the true incident spectrum from the low flux measurements. In the case of using non-paralyzable models, the RMSD and COV between the models and the measurements increase up to 43.30 and 26.2%, respectively. However, these values are still much lower than those between the measured spectrum and the true incident spectrum, 149.6, and 90.4%, respectively.

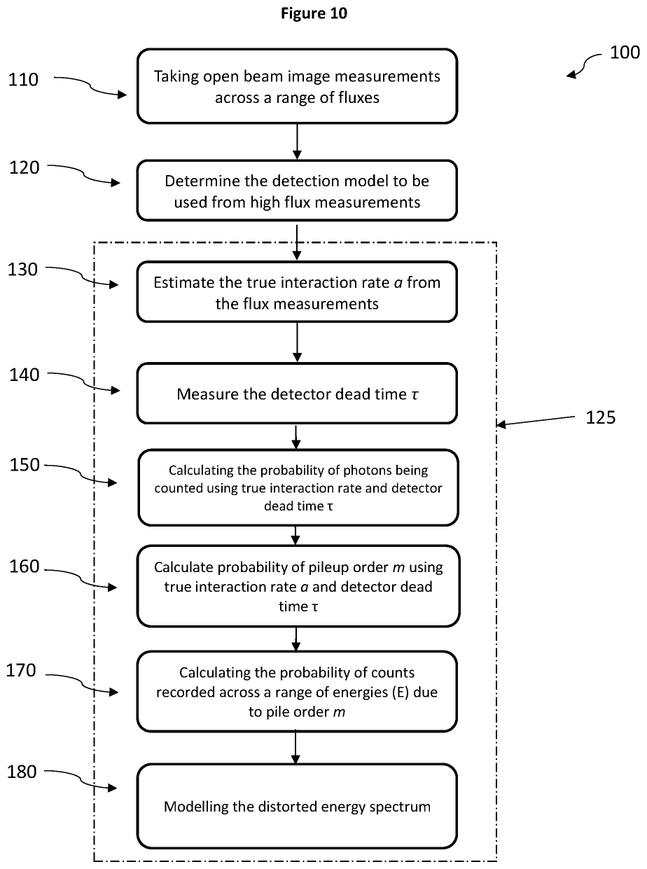

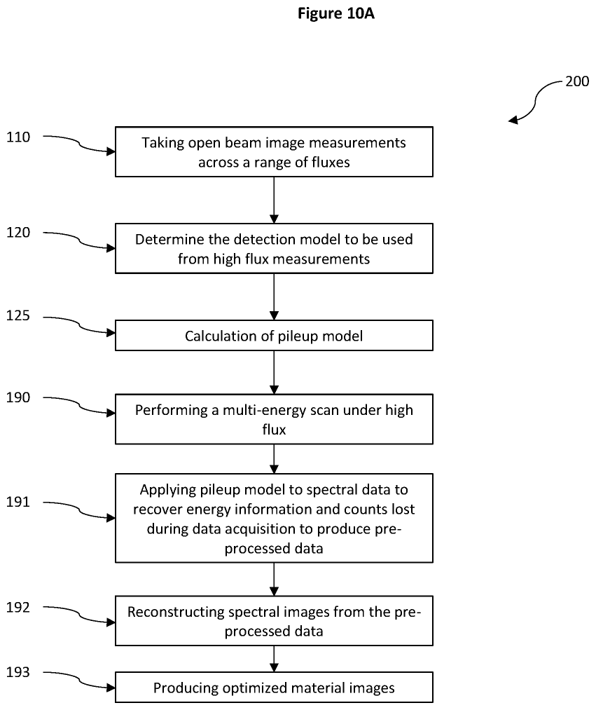

FIG. 10 shows the method step 100 of simulating a distorted energy spectrum in a spectral scanning system.

Open beam images 110 are acquired across a range of different photon fluxes, which may be controlled by the tube current settings, with an increase in the tube current producing higher fluxes.

Once open bean measurements are taken, the detection model used by the photon counting detector is determined 120. The counting mechanism of a photon counting detectors is modelled either by paralyzable or non-paralyzable detection models. However, subtle effects often make such detectors behave somewhere in between.

In a non-paralyzable detection model, after a photon turns the detector into the inactive state, the detector returns to the active state after a fixed dead time period τ. Therefore, the rate of incoming photons does not influence duration of the dead time in a non-paralyzable detection model.

In a paralyzable model, the detector dead time is not fixed, rather any additional photon arriving within the dead time of the previous event resets the dead time and keeps the detector in the inactive state for another t period.

The pulses generated in the analogue circuit of the Medipix detectors are bipolar. In addition, the energy of the observed event is proportional to the height of the pulse generated in the analogue circuit. The overlap of the pulses can occur in two ways. Peak pileup occurs when two or more events interact with the detector within a dead time interval. In this case, a positive lobe of a pulse overlaps the main positive lobes of the previous pulses. Since the detector is in the inactive state, the two pulses cannot be resolved separately. Therefore, several events are observed as one, with a registered energy higher than the energies of the original photon events.

The energy of the observed photon events may correspond to an energy above the x-ray tube voltage (kVp). Therefore, peak pileup leads to the distortion of the recorded energy spectrum by losing counts and adding a high energy tail.

Another type of pileup effect occurs when a positive lobe of a pulse overlaps the negative tails of the previous pulses. This effect is called tail pileup in which the overlaps can cause the energy of the photons to be observed slightly lower than their original energies. Therefore, tail pileup may add a small low energy tail to the measured spectrum. One should notice that although tail pileup distorts the energy spectrum, it may not influence the number of counts.

The true interaction rate 130 is estimated using the measurements derived at step 110. In order to study and understand the influence of the photon pileup effect, the spectrum that interacts with the sensor layer needs to be known. The true interaction rate is defined as the fraction of the photon flux that interacts with the sensor and contributes to the detector measurements. A higher interaction rate requires a faster photon processing circuit that allows resolving a photon before a subsequent photon interacts with the sensor. The true interaction rate is energy dependent and varies based on the quantum detection efficiency of the sensor and the filtration used for the measurements. These two factors can modulate the polychromatic source spectrum and reduce the number of photons that interact with the sensor. A filter attenuates the source spectrum and reduces the number of photons that reach the detector, therefore reduces the chance of photons to be piled up.

Once the true interaction rate a has been estimated, the detector dead time is measured 140 using one of a number of techniques discussed in more detail above. Following this, steps 150-180 demonstrate the calculation of probabilities of photons being counted, pileup order, the probability of counts recorded across a range of energies and the probability of counts across the energy spectrum due to every order of pileup respectively. For the purposes of this application, steps 130-180 are collectively referred to as pileup model calculation 125. The techniques and methods for making these calculations are discussed in further detail above.

FIG. 10A shows a method 200 for producing optimized material images using a multi-energy x-ray CT system, utilizing method 100 for modelling a distorted energy spectrum as shown in FIG. 10.

In method 10A, a pileup model is calculated using steps 110-125 as described in further detail above. A multi-energy scan is then performed under high flux of 7×10 190 to produce an initial set of scan data. The pileup model is then applied 191 to the scan data produced at step 190 by applying the pileup model to the initial scan data to produce an improved data set. This data set includes recovered information previously lost in the scanning step 190 due to the pileup effect. This improved data set is then used during reconstruction of the data set 192, the reconstructed data is then used to produce optimized images 193 of the material being scanned.

The optimised images produces multi-energy image scan of a quality that may typically only be generated using a scanning protocol with a much lower flux rate, over a significantly longer period of time. The application of the models of the present invention significantly decreases scan time, while retaining image quality.

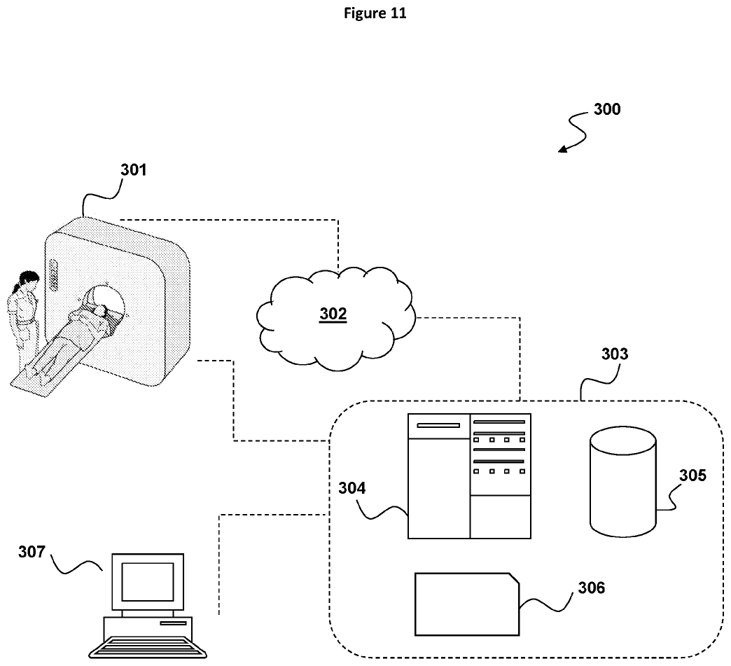

The methods for simulating a distorted energy spectrum as shown in method 100 in FIG. 10 may be implemented using a computer software program and carried out using the system as shown in FIG. 11 and described in further detail below.

In use the system 300 of FIG. 11 includes a spectral CT imaging system 301. Spectral CT images are taken of an object/sample or patient of interest using a detector system and the image data set delivered via communications network 302 to computer system 303. Computer system 303 includes processor 304, data storage medium 305 and server 306.

Processor 304 receives the image data set from communications network 302 and implements the processing steps of the methods of the present invention together with data storage medium 305. Alternatively, present invention may be implemented in the imaging system 301. Eventually, server 306 serves data from computer system 303 to interface 307, where optimized images or scan data may be displayed.

Interface 307 may be an application program interface (API), a user interface, software or hardware interface.

The various operations of methods described above may be performed by any suitable means capable of performing the operations, such as various hardware and/or software component(s), circuits, and/or module(s). Generally, any operations illustrated in the Figures may be performed by corresponding functional means capable of performing the same or equivalent operations.

The various illustrative modules and algorithm steps described in connection with the embodiments disclosed herein may be implemented as electronic hardware, computer software, or combinations of both. To clearly illustrate this interchangeability of hardware and software, various illustrative components, modules, and steps have been described above generally in terms of their functionality. Whether such functionality is implemented as hardware or software depends upon the particular application and design constraints imposed on the overall system. The described functionality may be implemented in varying ways for each particular application, but such implementation decisions should not be interpreted as causing a departure from the scope of the embodiments of the invention.

The steps of a method or algorithm and functions described in connection with the embodiments disclosed herein may be embodied directly in hardware, in a software module executed by a processor, or in a combination of the two. If implemented in software, the functions may be stored on or transmitted over as one or more instructions or code on a tangible, non-transitory computer-readable medium.

A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD ROM, or any other form of storage medium known in the art.

A storage medium is coupled to the processor such that the processor can read information from, and write information to, the storage medium. In the alternative, the storage medium may be integral to the processor. Disk and disc, as used herein, includes compact disc (CD), laser disc, optical disc, digital versatile disc (DVD), floppy disk and blu ray disc where disks usually reproduce data magnetically, while discs reproduce data optically with lasers.

Combinations of the above should also be included within the scope of computer readable media. The processor and the storage medium may reside in an ASIC. The ASIC may reside in a user terminal. In the alternative, the processor and the storage medium may reside as discrete components in a user terminal.

The models described herein provide a number of advantages over the current general knowledge. They may be customized for use with a range of photon counting detectors (as well as Medipix), including but not limited to high-z sensor materials such as CdTe and CZT. It should be appreciated that sensor material does not limit the operation of the methods described herein. In addition, the models can be used for recovery of spectral data of a wide range of pre-clinical and industrial imaging applications.

The developed pileup models allow optimization of the scan settings for the data acquisition.

Given the scan protocol and ability of the Medipix detectors in resolving the consecutive photons, the pileup models are capable of simulating the measured spectrum in the spectral scanning systems prior to the scan.

By simulating the contribution of the pileup effect to the measurements, the developed models allow the future revision of spectral scanning reconstruction calculations to cope with count loss and energy distortion.

Such compensations allow use of higher fluxes that results in shorter imaging times. Not only this will be beneficial to human spectral imaging, but also broadens applications of the spectral imaging in which short scanning time is required.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

1-21: (canceled)

22. A computer implemented method for modelling an energy spectrum distorted due to the pileup effect using probability distribution functions in a multi-energy x-ray CT imaging system, the method including the steps of:
a) receiving open beam spectrum measurements and count rate a_{R} measurements across a range of tube current settings Iₐ from 5 μA-400 μA and storing said data on a data storage medium;
b) determining the detection model from the count rate measurements of step a);
c) applying linear regression to the measurements at the lowest tube current settings from step a) to quantify a linear coefficient k which relates the true interaction rate a to tube currents Iₐ;
d) calculating the corresponding true interaction rate a for the tube currents Iₐ used for measurements in step a) from the linear coefficient k from step c);
e) approximating the detector dead time τ using a pre-defined probability function of photons being counted, count rates and corresponding true interaction rates from steps a) and d), and according to the detection model determined in step b);
f) selecting an exposure time setting tₑₓₚ of the imaging system and a tube current setting of Iₐ>200 μA;
g) calculating the true interaction rate a corresponding to the tube current setting selected in step f);
h) calculating the probability of photons being counted for the true interaction rate a calculated in step g) and the detector dead time τ from step d);
i) calculating the probability of m^{th} order of pileup using detector dead time τ and true interaction rate a from steps g) and d);
j) calculating probability distribution of photon energy across the energy spectrum for the tube current Iₐ from step f) and from the measured spectrum at the lowest tube current setting used from step a) and the linear coefficient k from step c);
k) calculating the probability of counts recorded across a range of energies (E) due to a range of orders of pileup, m; and
l) modelling the probability of counts across the distorted energy spectrum based on the outputs from steps a) to k).

23. The computer implemented method of claim 22, wherein the detection model of step (b) is a paralyzable or non-paralyzable detection model.

24. The computer implemented method of claim 22, wherein the step of quantifying a linear coefficient k at step c) includes applying linear regression to two or more measured count rates a_{R} from step a) acquired with tube current settings between 5-15 μA using the formula;
a=k×Ia

25. The computer implemented method of claim 22, wherein a set of true interaction rate values a is calculated for the set of tube currents Iₐ used in step a) using the linear equation from step c).

26. The computer implemented method of claim 23, wherein the probability of photons being counted for non-paralyzable and paralyzable detection models are calculated from either the formula;
$$P {r}_{r e c} ( aτ ) = \{ \frac{1}{aτ + 1}$$
for a non-paralyzable detection model; or
Pr_{rec}(aτ)={e^{-aτ}
for a paralyzable detection model. wherein
$${r}_{r e c} ( a τ ) = \frac{{a}_{R}}{a} .$$

27. The computer implemented method of claim 22, wherein the step of approximating the detector dead time τ at step d) uses regression to calculate a value for τ which best fits the probability function of photons being counted to a set of count rates a_{R} and a set of true interaction rates a from steps a) and d).

28. The computer implemented method of claim 22, wherein at step g) the true interaction rate corresponding to the tube current selected in step f) is calculated using the linear coefficient k from step c).

29. The computer implemented method of claim 22, wherein step i) of calculating the probability of m^{th} order of pileup using detector dead time τ and true interaction rate a from steps e) and g) uses either the formula;
$$P {r}_{m} ( aτ , m ) = \frac{{( aτ )}^{m} {e}^{- aτ}}{m !}$$
for a non-paralyzable detection model; or Prₘ(aτ,m)=(1−e^{-aτ})^{m}×e^{-aτ} for a paralyzable detection model. wherein m represents the pileup order.

30. The computer implemented method of claim 22, wherein step j) of calculating probability distribution of photon energy across the energy spectrum for the tube current Iₐ uses the formula;
$$P {r}_{γ} ( E ) = \frac{{S}_{0} ( E )}{{\int }_{0}^{∞} {S}_{0} ( E )}$$
wherein S₀(E) is the true incident spectrum, S₀(E) is calculated by multiplying the linear coefficient k from step c) by the measured spectrum from the lowest tube current setting from step a).

31. The computer implemented method as claimed in claim 22, wherein step k) of calculating the probability of counts recorded across a range of energies due to pile up of different orders is achieved using the formula;
$$Pr ( E , m ) = ( \frac{1}{m + 1} ) [ {Pr}_{γ} ( E ) {★}^{( m )} {Pr}_{γ} ( E ) ]$$
wherein ⋅^{(m)} represents the operation of convolution for m times.

32. The computer implemented method of claim 22, wherein the step I) of modelling the probability of counts across the distorted energy spectrum includes calculating the number of counts (Nₚᵤ) observed at energy (E) with a 1 keV interval by using the formula;
$${N}_{pu} ( E ) = a \times {t}_{exp} \times {Pr}_{rec} ( aτ ) \times {\sum }_{m = 0}^{∞} [ {Pr}_{m} ( aτ , m ) \times Pr ( E , m ) ]$$
wherein a is the true interaction rate, tₑₓₚ is the exposure time, τ is the detector dead-time, Pr_{rec}(aτ) is the probability of photons being counted, Pr_{rec}(aτ,m) is the probability of pulse pileup order m, and Pr(E,m) is the probability of the counts recorded across the energy spectrum with mₜₕ order of pileup, wherein pileup of order m occurs when m+1 photons interact with a detector pixel within the same dead time period τ.

33. The computer implemented method as claimed in claim 22, wherein the method models one or more distortions in an energy spectrum due to a peak pileup effect.

34. The computer implemented method as claimed in claim 22, wherein the method models one or more distortions in an energy spectrum due to a tail pileup effect.

35. A computer implemented method for optimising images produced by a multi-energy x-ray CT imaging system, the method including the step of incorporating the computer implemented method for simulating a distorted energy system as claimed in claim 22 into a multi-energy x-ray scanning and image reconstruction process.

36. The computer implemented method of claim 22, wherein the method is incorporated into, or used in conjunction with a spectral image reconstruction algorithm and a material decomposition algorithm.

37. A computer implemented method for producing optimised material images using a multi-energy x-ray CT imaging system, the method including modelling a distorted energy spectrum in a multi-energy x-ray CT imaging system using probability distribution functions, the method for modelling including the steps of;
a) receiving open beam spectrum measurements and count rate a_{R} measurements across a range of tube current settings Iₐ and storing said data on a data storage medium;
b) determining the detection model from the count rate measurements of step a);
c) applying linear regression to the measurements at the lowest tube current settings from step a) to quantify a linear coefficient k which relates the true interaction rate a to tube currents Iₐ;
d) calculating the corresponding true interaction rate a for the tube currents Iₐ used for measurements in step a) from the linear coefficient k from step c);
e) approximating the detector dead time τ using a pre-defined probability function of photons being counted, count rates and corresponding true interaction rates from steps a) and d), and according to the detection model determined in step b);
f) selecting an exposure time setting tₑₓₚ of the imaging system and a high tube current setting;
g) calculating the true interaction rate a corresponding to the tube current setting selected in step f);
h) calculating the probability of photons being counted for the true interaction rate a calculated in step g) and the detector dead time τ from step d);
i) calculating the probability of m^{th} order of pileup using detector dead time τ and true interaction rate a from steps g) and d);
j) calculating probability distribution of photon energy across the energy spectrum for the tube current Iₐ from step f) and from the measured spectrum at the lowest tube current setting used from step a) and the linear coefficient k from step c);
k) calculating the probability of counts recorded across a range of energies (E) due to a range of orders of pileup, m; and
l) modelling the probability of counts across the distorted energy spectrum based on the outputs from steps a) to k);
wherein the method for producing optimised images further comprises;
m) operating a multi-energy x-ray scan using under a high tube current to produce a set of scan data;
n) applying the model of step h) to the scan data produced at step m) to recover energy information and counts lost during acquisition of the scan data to produce a set of pre-processed data;
o) reconstructing spectral images from the pre-processed data in (n);
p) producing optimised material images.

38. A computer implemented method for modelling a distorted energy spectrum using probability distribution functions in a multi-energy x-ray CT imaging, the method including the steps of:
a) receiving open beam count rate measurements across a range of tube current settings and storing said data on a data storage medium;
b) determining the detection model from measurements of step a);
c) estimating the true interaction rate a from measurements made in step a);
d) approximating the detector dead time τ using count rates from step a) and corresponding true interaction rates from c), and according to the detection model determined in step b);
e) calculating the probability of photons being counted for any true interaction rate a, for a detection model determined from step b) and using the detector dead time τ from step d);
f) calculating the probability of m^{th} order of pileup using true interaction rate a and detector dead time τ from steps c) and d);
g) calculating the probability of counts recorded across a range of energies (E) due to pileup order m; and
modelling the probability of counts across the distorted energy spectrum based on the outputs from steps a) to g).
